(19) United States
(12) Patent Application Publication
(10) Pub. No.: US 2007/0276490 A1
(43) Pub. Date: Nov. 29, 2007

(54) Dynamic Spinal Plate Implant and Method of Use

(76) Inventor: Richard J. Mateyka, Las Vegas, NV (US)

Correspondence Address: PROCOPIO, CORY, HARGREAVES & SAVITCH LLP, 530 B STREET, SUITE 2100, SAN DIEGO, CA, 92101, US

(21) Appl. No.: 11/383,278

(22) Filed: May 15, 2006

(51) Int. Cl. A61F 2/44

(52) U.S. Cl. 623/17.11; 606/69

(57) ABSTRACT

A method of using a dynamic plate surgical implant includes removing a patient's degenerated disc between upper vertebrae and lower vertebrae; inserting an artificial replacement disc between the upper vertebrae and lower vertebrae where the degenerated disc was removed; attaching a dynamic plate surgical implant to an anterior of the upper vertebrae and the lower vertebrae, the dynamic plate surgical implant including an upper plate member and a lower plate member dynamically connected through a dynamic movement mechanism that allows extension, flexion, lateral flexion right an left, and rotation of the upper plate member relative to the lower plate member; and using the dynamic plate surgical implant to prevent the artificial replacement disc from kicking out anterior to the vertebrae while allowing the patient's spine to maintain its normal range of motion, reducing the risk of degeneration in adjacent segment of the spine.

### BACKGROUND

1. Field of the Invention

The present invention relates to cervical and lumbar plate surgical implants used during spinal surgery.

2. Related Art

Historically, ongoing low-back pain from degenerative low-back conditions that does not get better with non-surgical care has been treated with some type of lumbar spinal fusion surgery. During this procedure, anterior plate implants have been used in the cervical and lumbar regions during spinal fusion surgery. The anterior plate implants include an elongated plate that is screwed into vertebrae adjacent to the fused area to prevent relative motion. However, a problem with spine fusion is that it does change the mechanics of the segment of the low back that is fused. The anterior plate implants exacerbate this problem because they prevent relative movement of the vertebrae.

One major intended benefit of artificial disc replacement (ADR) surgery over spine fusion is that it does not change the biomechanics of the lower (lumbar) spine or the upper (cervical) spine, allowing the spine to maintain its normal range of motion and thereby reducing or eliminating the risk of degeneration in adjacent segments of the spine.

During artificial disc replacement surgery, the patient's degenerated disc is removed and a pair of endplates are inserted in the space between the vertebrae and attached to the vertebrae above and below the disc. A material (e.g., polyethylene) is then inserted between the plates to create a disc-like structure that mimics the normal disc by providing both a normal level of separation between the vertebrae and allowing the usual range of motion and flexibility for that segment of the spine.

### SUMMARY

The inventor of the present invention has recognized that what is needed in artificial disc replacement surgery is a dynamic plate surgical implant that attaches to the vertebrae adjacent to the artificial disc, allowing the spine to maintain its normal range of motion while preventing the disc replacement from kicking out anterior to the vertebrae.

Another aspect of the invention involves a method of using a dynamic plate surgical implant. The method includes removing a patient's degenerated disc between upper vertebrae and lower vertebrae; inserting an artificial replacement disc between the upper vertebrae and lower vertebrae where the degenerated disc was removed; attaching a dynamic plate surgical implant to an anterior of the upper vertebrae and the lower vertebrae, the dynamic plate surgical implant including an upper plate member and a lower plate member dynamically connected through a dynamic movement mechanism that allows extension, flexion, lateral flexion right an left, and rotation of the upper plate member relative to the lower plate member; and using the dynamic plate surgical implant to prevent the artificial replacement disc from kicking out anterior to the vertebrae while allowing the patient's spine to maintain its normal range of motion, reducing the risk of degeneration in adjacent segment of the spine.

An additional aspect of the invention involves a method of using a dynamic plate surgical implant during artificial replacement disc surgery where a patient's degenerated disc between upper vertebrae and lower vertebrae is replaced with an artificial replacement disc. The method includes attaching a dynamic plate surgical implant to an anterior of the upper vertebrae and the lower vertebrae, the dynamic plate surgical implant including an upper plate member and a lower plate member dynamically connected through a dynamic movement mechanism that allows extension, flexion, lateral flexion right an left, and rotation of the upper plate member relative to the lower plate member; and using the dynamic plate surgical implant to prevent the artificial replacement disc from kicking out anterior to the vertebrae while allowing the patient's spine to maintain its normal range of motion, reducing the risk of degeneration in adjacent segment of the spine.

Other features and advantages of the present invention will become more readily apparent to those of ordinary skill in the art after reviewing the following detailed description and accompanying drawings.

### BRIEF DESCRIPTION OF THEE DRAWINGS

The details of the present invention, both as to its structure and operation, may be gleaned in part by study of the accompanying drawings, in which like reference numerals refer to like parts, and in which:

FIG. 1 is a front elevational view or anterior view of an embodiment of a dynamic spinal plate implant used during artificial disc replacement surgery; and

FIG. 2 is a right side elevational view or lateral view of the dynamic spinal plate implant illustrated in FIG. 1, and shows the dynamic spinal plate implant applied to upper and lower vertebrae adjacent an artificial disc replacement.

### DETAILED DESCRIPTION

Certain embodiments as disclosed herein provide for a dynamic plate surgical implant and method of use that attaches to the vertebrae adjacent to an artificial replacement disc during artificial disc replacement surgery, allowing the spine to maintain its normal range of motion while preventing the disc replacement from kicking out anterior to the vertebrae.

After reading this description it will become apparent to one skilled in the art how to implement the invention in various alternative embodiments and alternative applications. However, although various embodiments of the present invention will be described herein, it is understood that these embodiments are presented by way of example only, and not limitation. As such, this detailed description of various alternative embodiments should not be construed to limit the scope or breadth of the present invention as set forth in the appended claims.

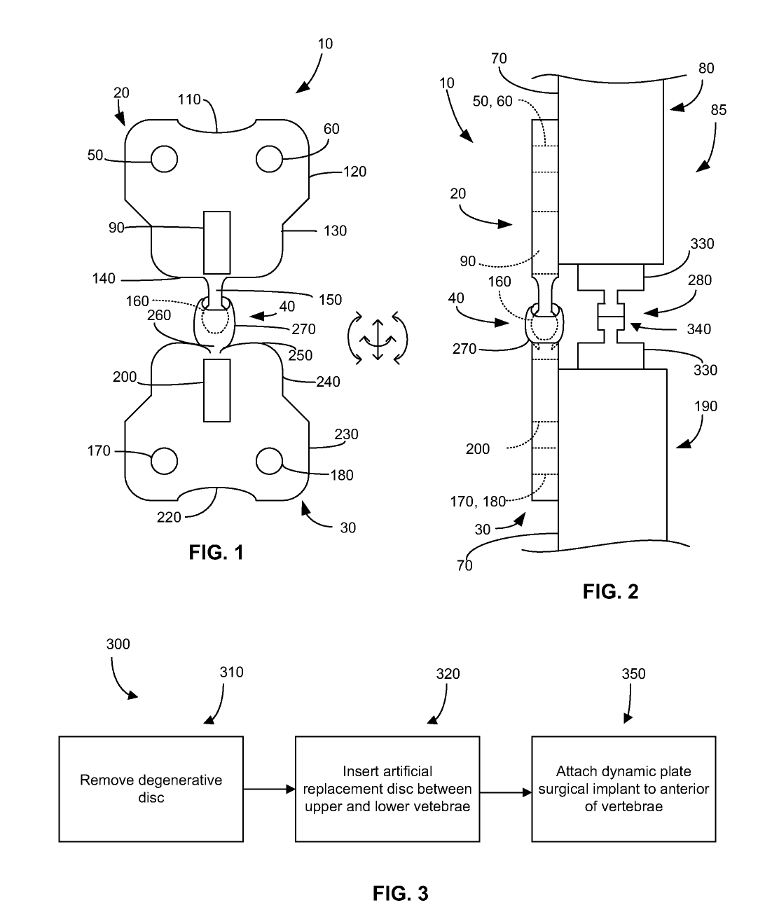

With reference to FIGS. 1 and 2, an embodiment of a dynamic plate surgical implant (“implant”) 10 will now be described. The implant 10 includes an upper plate member 20 and a lower plate member 30 rotatably and pivotally connected to each other through a dynamic movement mechanism 40. Although the dynamic movement mechanism 40 is shown and described in the embodiment as a ball-and-socket joint, in alternative embodiments, the dynamic movement mechanism 40 includes alternative mechanisms that provide the same or similar relative dynamic movement to that shown and described.

The upper plate member 20 includes a flat plate made of titanium. The upper plate member 20 includes first and second screw holes 50, 60 that receive screws (not shown) for attaching the plate 10 to an anterior side 70 of upper vertebrae 80 of vertebrae 85. The upper plate member 20 includes elongated rectangular window 90. The upper plate member 20 includes an incurved top 110 of a head portion 120. The head portion 120 includes the screw holes 50, 60. The head portion 120 tapers down in width into connecting portion 130. Extending longitudinally outward from a bottom 140 of the connecting portion 130 is a neck 150 terminating in a swivel ball 160.

The lower plate member 30 includes a flat plate made of titanium. The lower plate member 30 includes first and second screw holes 170, 180 that receive screws (not shown) for attaching the plate to an anterior side 70 of lower vertebrae 190. The lower plate member 30 includes elongated rectangular window 200. The lower plate member 30 includes an incurved top 220 of a head portion 230. The head portion 230 includes the screw holes 170, 180. The head portion 230 tapers down in width into connecting portion 240. Extending longitudinally outward from a bottom 250 of the connecting portion 240 is a neck 260 terminating in a socket 270 that receives the swivel ball 160.

With reference additionally to FIG. 3, a method 300 of using the dynamic plate surgical implant 10 will now be described. During artificial disc replacement surgery in the cervical and/or lumbar regions, at step 310, the patient's degenerated disc is removed. At step 320, the artificial replacement disc 280 is inserted between the upper vertebrae 85 and the lower vertebrae 190 to create a disc-like structure that mimics the normal disc by providing both a normal level of separation between the vertebrae and allowing the usual range of motion and flexibility for that segment of the spine. In an exemplary method, step 320 includes inserting a pair of endplates 330 in the space between the vertebrae 85, 190, and attaching the endplates 330 to the vertebrae 85, 190 above and below the artificial replacement disc 280. A material (e.g., polyethylene) 340 is then inserted between the plates 330 to create a disc-like structure that mimics the normal disc. At step 350, the dynamic plate surgical implant 10 is attached to the anterior 70 of the vertebrae 85, 190 adjacent to the artificial replacement disc 280. The upper plate 20 is positioned against the anterior 70 of the upper vertebrae 85 and the lower plate 30 is positioned against the anterior of the lower vertebrae 190 so that the dynamic movement mechanism 40 is substantially centered with respect to the artificial replacement disc 280, adjacent to and anterior of the artificial replacement disc 280. In this position, a lower portion of the vertebrae 85 and an upper portion of the artificial replacement disc 280 are viewable through the window 90. Similarly, in this position, an upper portion of the vertebrae 190 and a lower portion of the artificial replacement disc 280 are viewable through the window 200. Being able to view the vertebrae 80, 90, and artificial replacement disc 280 through the windows 90, 200 assists in placement and attachment of the dynamic plate surgical implant 10. Screws (not shown) are inserted through screw holes 50, 60, 170, 180 and screwed into the anterior 70 sides, into the upper vertebrae 85 and the lower vertebrae 190.

In this position, the dynamic plate surgical implant 10 prevents the artificial replacement disc 280 from kicking out anterior to the vertebrae 80. The dynamic movement mechanism 40 allows the plates 20, 30 to dynamically rotate and pivot freely with respect to each other so that the spine can maintain its normal range of motion. As shown by the arrows in FIG. 1, the dynamic movement mechanism 40 allows for the following movement: extension, flexion, lateral flexion right and left, and rotation. As a result, the biomechanics of the spine are not changed with the disc replacement surgery. The spine is allowed to maintain its normal range of motion, which reduces or eliminates the risk of degeneration in adjacent segments of the spine.

For multi-level disk replacement, the dynamic plate surgical implant 10 would continue in the same pattern as shown in FIG. 2 and described above. In one embodiment, for multi-level disk replacement, a separate dynamic plate surgical implant 10 is used for each artificial replacement disc 280. In an alternative embodiment, the dynamic plate surgical implant 10 includes multiple plates attached by dynamic movement mechanisms 40. For example, but not by way of limitation, if two adjacent disks are replaced, a three-plate dynamic plate surgical implant 10 is used, if three adjacent disks are replaced, a four-plate dynamic plate surgical implant 10 is used, if four adjacent disks are replaced, a five-plate dynamic plate surgical implant 10 is used, etc.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly limited by nothing other than the appended claims.

1. A method of using a dynamic plate surgical implant, comprising:
removing a patient's degenerated disc between upper vertebrae and lower vertebrae;
inserting an artificial replacement disc between the upper vertebrae and lower vertebrae where the degenerated disc was removed;
attaching a dynamic plate surgical implant to an anterior of the upper vertebrae and the lower vertebrae, the dynamic plate surgical implant including an upper plate member and a lower plate member dynamically connected through a dynamic movement mechanism that allows extension, flexion, lateral flexion right an left, and rotation of the upper plate member relative to the lower plate member;
using the dynamic plate surgical implant to prevent the artificial replacement disc from kicking out anterior to the vertebrae while allowing the patient's spine to maintain its normal range of motion, reducing the risk of degeneration in adjacent segment of the spine.

2. The method of claim 1, wherein attaching a dynamic plate surgical implant to an anterior of the upper vertebrae and the lower vertebrae includes attaching the upper plate member to the anterior of the upper vertebrae with one or more screws, and attaching the lower plate member to the anterior of the lower vertebrae with one or more screws.

3. The method of claim 1, wherein the dynamic movement mechanism is a ball-and-socket joint.

4. The method of claim 1, wherein the dynamic plate surgical implant is used in the cervical region.

5. The method of claim 1, wherein the dynamic plate surgical implant is used in the lumbar region.

6. The method of claim 1, wherein attaching a dynamic plate surgical implant to an anterior of the upper vertebrae and the lower vertebrae includes attaching the dynamic plate surgical implant to the anterior of the upper vertebrae and the lower vertebrae so that the dynamic movement mechanism is substantially centered between the upper vertebrae and the lower vertebrae, anterior of the artificial replacement disc.

7. The method of claim 1, wherein inserting an artificial replacement disc between the upper vertebrae and lower vertebrae includes inserting a pair of endplates in space between the upper vertebrae and the lower vertebrae, attaching the endplates to the upper vertebrae and lower vertebrae, providing a material between the endplates to create a disc-like structure that mimics a normal disc.

8. The method of claim 1, wherein attaching a dynamic plate surgical implant to an anterior of the upper vertebrae and the lower vertebrae includes viewing the upper vertebrae and the artificial replacement disc through an upper window of the dynamic plate surgical implant during the attaching step, and viewing the lower vertebrae and the artificial replacement disc through a lower window of the dynamic plate surgical implant during the attaching step.

9. The method of claim 1, wherein the dynamic plate surgical implant is used for multi-level disk replacement.

10. A method of using a dynamic plate surgical implant during artificial replacement disc surgery where a patient's degenerated disc between upper vertebrae and lower vertebrae is replaced with an artificial replacement disc, comprising:
attaching a dynamic plate surgical implant to an anterior of the upper vertebrae and the lower vertebrae, the dynamic plate surgical implant including an upper plate member and a lower plate member dynamically connected through a dynamic movement mechanism that allows extension, flexion, lateral flexion right an left, and rotation of the upper plate member relative to the lower plate member;
using the dynamic plate surgical implant to prevent the artificial replacement disc from kicking out anterior to the vertebrae while allowing the patient's spine to maintain its normal range of motion, reducing the risk of degeneration in adjacent segment of the spine.

11. The method of claim 10, wherein attaching a dynamic plate surgical implant to an anterior of the upper vertebrae and the lower vertebrae includes attaching the upper plate member to the anterior of the upper vertebrae with one or more screws, and attaching the lower plate member to the anterior of the lower vertebrae with one or more screws.

12. The method of claim 10, wherein the dynamic movement mechanism is a ball-and-socket joint.

13. The method of claim 10, wherein the dynamic plate surgical implant is used in the cervical region.

14. The method of claim 10, wherein the dynamic plate surgical implant is used in the lumbar region.

15. The method of claim 10, wherein attaching a dynamic plate surgical implant to an anterior of the upper vertebrae and the lower vertebrae includes attaching the dynamic plate surgical implant to the anterior of the upper vertebrae and the lower vertebrae so that the dynamic movement mechanism is substantially centered between the upper vertebrae and the lower vertebrae, anterior of the artificial replacement disc.

16. The method of claim 10, wherein attaching a dynamic plate surgical implant to an anterior of the upper vertebrae and the lower vertebrae includes viewing the upper vertebrae and the artificial replacement disc through an upper window of the dynamic plate surgical implant during the attaching step, and viewing the lower vertebrae and the artificial replacement disc through a lower window of the dynamic plate surgical implant during the attaching step.

17. The method of claim 10, wherein the dynamic plate surgical implant is used for multi-level disk replacement.
